(19) United States
(12) Patent Application Publication
(10) Pub. No.: US 2016/0015646 A1
(43) Pub. Date: Jan. 21, 2016

(54) Oral delivery system for sorafenib tosylate

(71) Applicant: David Wong, Milpitas, CA (US)

(72) Inventor: David Wong, Milpitas, CA (US)

(73) Assignee: David Wong, Milpitas, CA (US)

(21) Appl. No.: 14/333,735

(22) Filed: Jul. 17, 2014

(51) Int. Cl. A61K 9/20; A61K 31/44

(52) U.S. Cl. CPC A61K 9/2072; A61K 31/44; A61K 9/2027

(57) ABSTRACT

The present invention relates to a gastroretentive tablet for treating unresectable hepatocellular carcinoma, comprising an enteric polymer, a nanoparticle and an excipient, wherein the nanoparticle comprises an oral multikinase inhibitor, wherein the oral multikinase inhibitor is coated with an amino methacrylate copolymer, wherein the oral multikinase inhibitor is sorafenib, and wherein the enteric polymer is methacrylic acid copolymer. The excipient is selected from a group consisting of a retarding agent, a binder, a filler, a diluent, a disintegrant, a lubricant, a colorant, a solubilizing agent, or a mixture thereof.

### TECHNICAL FIELD

The present invention relates to a gastroretentive tablet for treating unresectable hepatocellular carcinoma, comprising an enteric polymer, a nanoparticle and an excipient, wherein the nanoparticle comprises an oral multikinase inhibitor, wherein the oral multikinase inhibitor is coated with an amino methacrylate copolymer, wherein the oral multikinase inhibitor is sorafenib, and wherein the enteric polymer is methacrylic acid copolymer. The excipient is selected from a group consisting of a retarding agent, a binder, a filler, a diluent, a disintegrant, a lubricant, a colorant, a solubilizing agent, or a mixture thereof.

### BACKGROUND OF THE INVENTION

Hepatocellular carcinoma is a common type of liver cancer. Most cases of hepatocellular carcinoma are secondary to either a viral hepatitis infection or cirrhosis. In general, only 10-20% of hepatocellular carcinomas can be removed completely using surgery. If the cancer cannot be completely removed, the disease is usually deadly within a few months. Sorafenib tosylate tablet (marketed under the brand name of NEXAVAR®) was approved by the US FDA in 2005 to treat advanced hepatocellular carcinoma. Sorafenib tosylate is a kinase inhibitor, but it is very different from any other kinase inhibitors. Sorafenib targets both proliferation and angiogenesis by inhibiting c-KIT and Flt-3 on one hand; and vascular endothelial growth factor receptor (VEGFR) and platelet-derived growth factor receptor (PDGFR) on the other hand. C-KIT is a protein found on the surface of many different types of cells. It binds to a substance called stem cell factor (SCF), which causes certain types of blood cells to grow. C-kit may also be found in higher than normal amounts, or in a changed form, on some types of cancer cells. While, FLT3 is a receptor tyrosine kinase with important roles in hematopoietic stem/progenitor cell survival and proliferation. In addition, sorafenib also inhibits the serine/threonine RAF/MEK/ERK pathway. The Raf-MEK-ERK pathway is a key downstream effector of the Ras small GTPase, the most frequently mutated oncogene in human cancers. Thus, sorafenib tosylate is considered as a multikinase inhibitor. In the case where several kinases are overexpressed and several appear to contribute to the carcinogenesis, then a single multikinase inhibitor would be most effective. [Fleur Broekman et al, World J. Clin Oncol 2011 Feb. 10; 2(2): 80-93] Sorafenib tosylate is a white to yellowish or brownish solid. It is practically insoluble in aqueous media, slightly soluble in ethanol and soluble in PEG 400. NEXAVAR® is a conventional tablet; its excipients are croscarmellose sodium, microcrystalline cellulose, hypromellose, sodium lauryl sulfate, magnesium stearate, polyethylene glycol, titanium dioxide and ferric oxide red. NEXAVAR® comes with various adverse reactions. The most common adverse reactions (20%), which were considered to be related to NEXAVAR®, in patients with unresectable hepatocellular carcinoma or renal cell carcinoma are fatigue, weight loss, rash/desquamation, hand-foot skin reaction, alopecia, diarrhea, anorexia, nausea and abdominal pain. Further, in the clinical studies, the incidence of cardiac ischemia/infarction was 2.7% in NEXAVAR-treated patients compared with 1.3% in the placebo-treated group and in RCC Study 1, the incidence of cardiac ischemia/infarction was higher in the NEXAVAR-treated group (2.9%) compared with the placebo-treated group (0.4%). Consequently, it may be beneficial to have an alternative composition for sorafenib tosylate.

### BRIEF SUMMARY OF THE INVENTION

David Wong, the applicant and inventor of the current invention, has invented a novel tablet composition comprising sorafenib tosylate for treating unresectable hepatocellular carcinoma and renal cell carcinoma. And, the novel tablet composition is a pH dependent gastric retentive tablet.

Accordingly, in one aspect, the present invention relates to a novel gastric retentive tablet composition for treating unresectable hepatocellular carcinoma and renal cell carcinoma comprising sorafenib tosylate, an amino methacrylate copolymer, and an excipient, wherein the excipient is selected from a group consisting of a retarding agent, a binder, a filler, a diluent, a disintegrant, a lubricant, a colorant, a solubilizing agent, or a mixture thereof.

In a further aspect, the present invention relates to a novel gastric retentive tablet composition, wherein the drug particle is first coated with an amino methacrylate copolymer, and then mixed with other excipients, compressed into a tablet. The coat may further comprise a chelating agent.

### DETAILED DESCRIPTION OF THE INVENTION

### Definitions

“Optional” or “optionally” means that the subsequently described circumstance may or may not occur, so that the description includes instances where the circumstance occurs and instances where it does not.

Singular forms included in the claims such as “a”, “an” and “the” include the plural reference unless expressly stated or the context clearly indicates otherwise.

By “pharmaceutically acceptable” is meant a carrier comprised of a material that is not biologically or otherwise undesirable.

The term “gastric retentive tablet” refers to a tablet which is able to stay in the stomach for 2-4 hours. Tablet dimensions determine if it is a gastric retentive tablet; usually a tablet with a width of 10 mm shows gastric retention. (U.S. Pat. No. 8,377,453) In this invention, the width of the tablet is about 10 mm or longer, thus, it is a gastric retentive tablet and it is also an oral pharmaceutical tablet.

The term “nanoparticle” refers to particle with an average diameter less than 500 nm. The specification of the diameter is important, as it affects adsorption for most drugs directly.

### The Invention

The present invention provides a gastric retentive tablet composition and methods for preparing such composition. The drug is co-dissolved with an acid-soluble polymer in a solvent, spray-dried into a nanoparticle, then mixed with other excipients, optionally including an enteric polymer, then compressed into a tablet. Alternatively, the drug is suspended in an acid-soluble polymer solution (optionally comprising a chelator), spray-dried into a nanoparticle, then mixed with other excipients, optionally with an enteric polymer, then compressed into a tablet. The tablet is optionally coated for moisture barrier, taste-masking and/or cosmetic purposes. The gastric retentive tablet may have one or more of the following characteristics: (1) the tablet width is larger than 9.0 mm and (2) the tablet may swell in an aqueous medium.

The tablet can be formed by direct compression, granulation-compression, pellet-compression or equivalent methods. In direct compression, a “drug nanoparticle” and other excipients are well-mixed and placed in a press die, compressed to form a tablet. In granulation, a binder solution is sprayed onto a mixture of a “drug nanoparticle” and excipients to form granules. The granules are dried and milled to a desired particle size distribution. Then the granules are blended with other excipients, and placed in the press-die, compressed to form a tablet. Techniques for making tablets are described in Remington's Pharmaceutical Sciences, (Arthur Osol, editor), 1553-1593 (1980). Spray drying is the most popular method used in commercial production for nanoparticles, and is described in U.S. Pat. No. 8,287,764.

To achieve gastric retention, the width of an oval tablet needs to be larger than 10 mm and a proper tablet erosion rate is required. Thus, the tablet width in the invention has a width larger than 10 mm.

Accordingly, the present invention provides a gastroretentive tablet composition for treating unresectable hepatocellular carcinoma, comprising methacrylic acid copolymer, a nanoparticle and an excipient, wherein the nanoparticle comprises a core and an outer layer, wherein the core comprises an oral multikinase inhibitor, wherein the outer layer comprises an amino methacrylate copolymer, wherein the oral multikinase inhibitor is capable to inhibit c-KIT. Flt-3, VEGFR, PDGFR, and the RAF/MEK/ERK pathway, and wherein the excipient is selected from a group consisting of a retarding agent, a binder, a filler, a diluent, a disintegrant, a lubricant, a colorant, a chelating agent, a solubilizing agent, or a mixture thereof. In one aspect of this embodiment, the multikinase inhibitor is sorafenib tosylate.

In one embodiment, the gastroretentive tablet composition for treating unresectable hepatocellular carcinoma, comprising methacrylic acid copolymer, a nanoparticle and an excipient, wherein the nanoparticle comprises a core and an outer layer, wherein the core comprises an oral multikinase inhibitor, wherein the outer layer comprises an amino methacrylate copolymer, wherein the oral multikinase inhibitor is capable to inhibit c-KIT, Flt-3, VEGFR, PDGFR, and the RAF/MEK/ERK pathway, and wherein the excipient is selected from a group consisting of a retarding agent, a binder, a filler, a diluent, a disintegrant, a lubricant, a colorant, a chelating agent, a solubilizing agent, or a mixture thereof, and wherein the oral multikinase inhibitor is sorafenib tosylate. In one aspect of this embodiment, the gastroretentive tablet composition is a bilayer tablet, wherein both layers of the tablets are not “immediate-disintegrating layers”. In another aspect, the gastroretentive tablet is also able to treat renal cell carcinoma.

In another embodiment, the gastroretentive tablet composition for treating unresectable hepatocellular carcinoma, comprising methacrylic acid copolymer, a nanoparticle and an excipient, wherein the nanoparticle comprises a core and an outer layer, wherein the core comprises an oral multikinase inhibitor, wherein the outer layer comprises an amino methacrylate copolymer and a chelating agent, wherein the oral multikinase inhibitor is capable to inhibit c-KIT, Flt-3, VEGFR, PDGFR, and the RAF/MEK/ERK pathway, and wherein the excipient is selected from a group consisting of a retarding agent, a binder, a filler, a diluent, a disintegrant, a lubricant, a colorant, a solubilizing agent, or a mixture thereof, and wherein the oral multikinase inhibitor is sorafenib tosylate. In one aspect of this embodiment, the gastroretentive tablet composition is a bilayer tablet, wherein both layers of the tablets are not “immediate-disintegrating layers”. In another aspect, the gastroretentive tablet is also able to treat renal cell carcinoma.

In a particular embodiment, the gastroretentive tablet composition for treating unresectable hepatocellular carcinoma , comprising methacrylic acid copolymer, a nanoparticle and an excipient, wherein the nanoparticle comprises an oral multikinase inhibitor, a chelating agent, a solubilizing agent and an amino methacrylate copolymer, wherein the oral multikinase inhibitor is sorafenib tosylate, and wherein the excipient is selected from a group consisting of a retarding agent, a binder, a filler, a diluent, a disintegrant, a lubricant, a colorant, or a mixture thereof. In one aspect of this embodiment, the gastroretentive tablet composition is a bilayer tablet, wherein both layers of the tablets are not “immediate-disintegrating layers”. In another aspect, the gastroretentive tablet is also able to treat renal cell carcinoma.

The amount of excipient employed will depend upon how much active agent is to be used. One excipient can perform multi-functionally. Examples of excipients include but not limited to a retarding agent, a binder, a chelating agent, a filler, a diluent, a disintegrant, a lubricant, a solubilizing agent, a colorant, a chelating agent or a mixture thereof.

Enteric polymer is a polymer soluble at pH 5.5 or above. Examples of enteric polymer include but not limited to methacrylic acid copolymer, Type A, methacrylic acid copolymer, Type B, hydroxypropyl methylcellulose acetate succinate (also known as hypromellose acetate succinate), cellulose acetate phthalate, hydroxypropyl methyl cellulose phthalate, polyvinyl acetate phthalate, alginic acid, and sodium alginate. The preferred enteric polymer is methacrylic acid copolymer, Type A, NF.

Acid-soluble polymer is a water-soluble polymer, and dissolves in an aqueous medium regardless the environmental pH. In the embodiments of the invention, the acid-soluble polymer is not soluble at a pH higher than 6, The preferred acid-soluble polymer in the embodiments is amino methacrylate copolymer ((e.g. marketed under the brand name of EUDRAGIT® E).

Retarding material is a material retarding the drug release or slowing down the matrix erosion. Examples of retarding materials include, but are not limited to, hydroxyalkyl celluloses such as hydroxypropyl cellulose, hydroxypropylmethyl cellulose (2208, 2906 and 2910) or hydroxyethyl cellulose; polyvinyl derivatives such as povidone, crospovidone or polyvinyl alcohol; polyethylene oxides; methyl cellulose; gelatin; polysaccharides such as pregelatinized starch, partially pregelatinized starch, pullulan, dextrin, sodium alginate or gum Arabic, polyethylene glycols and some water-insoluble materials. In the invention, some embodiments specify polyethylene oxide. In fact, polyethylene oxide can be replaced with any high molecular weight polymers, preferably, a water soluble and water-swellable polymer.

Binders include, but are not limited to, starches such as potato starch, wheat starch, corn starch; microcrystalline cellulose; celluloses such as hydroxypropyl cellulose, hydroxyethyl cellulose, hydroxypropyl methylcellulose, ethyl cellulose, sodium carboxy methylcellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, which include, but are not limited to sugar, dextrates, dextrin, dextrose, fructose, lactitol, mannitol, sucrose, starch, lactose, xylitol, sorbitol, talc, microcrystalline cellulose, calcium salts such as carbonate, citrate, chloride, sulfate, phosphate; dibasic or tribasic, and the like can be used.

Lubricants may be selected from, but are not limited to, those conventionally known in the art such as magnesium, aluminum or calcium or zinc stearate, polyethylene glycol, glycerol monostearate, glyceryl monosterate, glyceryl behenate, mineral oil, sodium stearyl fumarate, stearic acid, hydrogenated vegetable oils and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The solubilizing agents include, but are not limited to, a surfactant, such as, for example, polysorbate 80 (Tween 80) and the like, a complexing agent, such as, for example, beta-cyclodextrins and the like, a polymer, such as, for example, poloxamer 188, and the like, a co-solvent, such as, for example, methanol and the like. The solubilizing agent may also be an acid or an alkaline, if the solubility of the drug is pH dependent.

Colorants include, but are not limited to, pharmaceutical grade dyes and pigments, red ferric oxide, yellow ferric oxide, titanium dioxide, carbon black, and indigo.

Disintegrants include, but are not limited to, crospovidone, croscarmellose-sodium, sodium starch glycolate, low-substituted hydroxypropylcellulose and other materials known to one of ordinary skill in the art.

Chelating agents, or chelators, include, but are not limited to, sodium benzoate, butylated hydroxytoluene, butylated hydroxyanisole and ethylenediaminetetraacetic acid.

The pharmaceutical dosage form of the invention can optionally have one or more coatings such as moisture-barrier film coating, sugar coating and other coatings known in the art. Coating is not considered as a matrix in this invention.

These coating layers comprises one or more excipients selected from the group comprising coating agents, plasticizers, channeling agents, opacifiers, taste-masking agents, fillers, polishing agents, coloring agents, anti-tacking agents and the like.

Coating agents which are useful in the coating process, include, but not limited to, polysaccharides such as maltodextrin, alkyl celluloses such as methyl or ethyl cellulose, cellulose acetate, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, OPADRY® coating systems, polyvinyl alcohol (PVA), copolymers of vinylpyrrolidone and vinyl acetate (e.g. marketed under the brand name of PLASDONE®) and polymers based on methacrylic acid such as those marketed under the brand name of EUDRAGIT®. These may be applied from aqueous or non-aqueous systems or combinations of aqueous and non-aqueous systems as appropriate.

Additives can be included along with the film formers to obtain satisfactory films. These additives can include plasticizers such as dibutyl phthalate, triethyl citrate, polyethylene glycol (PEG) and the like, channeling agents such as surfactants, short-chain water-soluble polymers, salts and the like, anti-tacking agents such as talc, stearic acid, magnesium stearate and colloidal silicon dioxide and the like, fillers such as talc, precipitated calcium carbonate, polishing agents such as Beeswax, carnauba wax, synthetic chlorinated wax and opacifying agents such as titanium dioxide and the like. All these excipients can be used at levels well known to the persons skilled in the art.

### EXAMPLES OF INVENTION

The foregoing examples are illustrative embodiments of the invention and are merely exemplary. A person skilled in the art may make variations and modifications without deviating from the spirit and scope of the invention. All such modifications and variations are intended to be included within the scope of the invention.

### Example 1

Sorafenib tosylate, 400 mg, is mixed with an amino methacrylate copolymer (200 mg) solution, then spray-dried to form nanoparticles. The drug nanoparticle, 600 mg, is mixed with methacrylic acid copolymer, 90 mg, polyethylene oxide, microcrystalline cellulose 400 mg and glycerol monostearate 20 mg, and then compressed into a tablet.

### Example 2

Sorafenib tosylate, 200 mg, is mixed with amino methacrylate copolymer, 100 mg and a chelating agent, 10 mg, in 0.01 N HCl, then spray-dried to form drug nanoparticles. The drug nanoparticle, 400 mg, is mixed with uncoated drug particle, 200 mg, methacrylic acid copolymer, 90 mg, polyethylene oxide, 50 mg, microcrystalline cellulose 400 mg and glycerol monostearate 20 mg, and then compressed into a tablet.

1. A gastroretentive tablet composition for treating unresectable hepatocellular carcinoma, comprising methacrylic acid copolymer, Type A, NF, a nanoparticle and an excipient, wherein the nanoparticle comprises a core and an outer layer, wherein the core comprises an oral multikinase inhibitor, wherein the outer layer comprises an amino methacrylate copolymer, wherein the oral multikinase inhibitor is capable to inhibit c-KIT, Flt-3, VEGFR, PDGFR, and the RAF/MEK/ERK pathway, wherein the amino methacrylate copolymer is an acid soluble polymer, wherein the amino methyacrylate copolymer is not soluble at a pH higher than 6, and wherein the excipient is selected from a group consisting of a retarding agent, a binder, a filler, a diluent, a disintegrant, a lubricant, a colorant, a chelating agent, a solubilizing agent, or a mixture thereof.

2. A gastroretentive tablet composition for treating unresectable hepatocellular carcinoma, comprising methacrylic acid copolymer, Type A, NF, a nanoparticle and an excipient, wherein the nanoparticle comprises a core and an outer layer, wherein the core comprises an oral multikinase inhibitor, wherein the outer layer comprises an amino methacrylate copolymer, wherein the oral multikinase inhibitor is capable to inhibit c-KIT, Flt-3, VEGFR, PDGFR, and the RAF/MEK/ERK pathway, wherein the amino methacrylate copolymer is an acid soluble polymer, wherein the amino methyacrylate copolymer is not soluble at a pH higher than 6, and wherein the excipient is selected from a group consisting of a retarding agent, a binder, a filler, a diluent, a disintegrant, a lubricant, a colorant, a chelating agent, a solubilizing agent, or a mixture thereof, and wherein the oral multikinase inhibitor is sorafenib tosylate.

3. A gastroretentive tablet composition for treating unresectable hepatocellular carcinoma, comprising methacrylic acid copolymer, Type A, NF, a nanoparticle and an excipient, wherein the nanoparticle comprises a core and an outer layer, wherein the core comprises an oral multikinase inhibitor, wherein the outer layer comprises an amino methacrylate copolymer and a chelating agent, wherein the amino methacrylate copolymer is an acid soluble polymer, wherein the amino methyacrylate copolymer is not soluble at a pH higher than 6, wherein the oral multikinase inhibitor is capable to inhibit c-KIT, Flt-3, VEGFR, PDGFR, and the RAF/MEK/ERK pathway, and wherein the excipient is selected from a group consisting of a retarding agent, a binder, a filler, a diluent, a disintegrant, a lubricant, a colorant, a solubilizing agent, or a mixture thereof, and wherein the oral multikinase inhibitor is sorafenib tosylate.
